(19) United States
(12) Patent Application Publication
(10) Pub. No.: US 2026/0079100 A1
(43) Pub. Date: Mar. 19, 2026

(54) OIL DIAGNOSTIC METHOD AND OIL DIAGNOSTIC SYSTEM

(71) Applicant: Hitachi, Ltd., Tokyo (JP)

(72) Inventor: Kyoko KOJIMA, Tokyo (JP)

(73) Assignee: Hitachi, Ltd., Tokyo (JP)

(21) Appl. No.: 19/108,406

(22) PCT Filed: Sep. 12, 2023
(86) PCT No.: PCT/JP2023/033164

§ 371 Date: Mar. 3, 2025

(30) Foreign Application Priority Data
Nov. 15, 2022 (JP) 2022-182880

(51) Int. Cl. G01N 21/27; G01N 33/30

(52) U.S. Cl. CPC G01N 21/27; G01N 33/30

(57) ABSTRACT

The purpose of the present invention is to acquire a uniform standard value for assessing oil property using an optical sensor. An oil diagnostic method according to an aspect of the present invention includes: storing, in a storage device, a calibration curve based on first data and second data, the first data indicating, as a color scale value, a color change of a standard substance having a known color scale value, and the second data indicating a color change based on a result of measuring the standard substance by using an optical sensor; acquiring, from an input device, third data based on a result of measuring oil having unknown color coordinates by using the optical sensor; and calculating, by using a processing device, a color scale value corresponding to the color coordinates of the oil from the third data by referring to the calibration curve.

### TECHNICAL FIELD

The present invention relates to techniques for assessment of the remaining lifetime of liquid typically represented by oil, and condition monitoring of machinery.

### BACKGROUND ART

A technique for diagnosing properties of lubricating oil used for rotary parts such as bearings and gears is essential in terms of maintenance and preservation of a large-scale rotary machine. Examples of the large-scale rotary machine include an amplifier for wind generator, an air compressor, a ship, and a power generation turbine. Oil is used for a transformer except the rotary machine. Examples of oil (oil: a hydrophobic chemical product having a phase separated from water) for machinery include the lubricating oil and grease (lubricant brought into a semi-solid state by adjusting viscosity of the lubricating oil).

Types of lubricating oil in accordance with purpose of use include engine oil, turbine oil, hydraulic oil, bearing oil, sliding surface oil, gear oil, compressor oil, cutting oil, and the like. In order to satisfy required performances, the lubricating oil of the respective types is blended with various additives.

As a trend in recent years, the machine condition monitoring has been performed based on the strategy aiming at minimum life cycle cost of the machine. Large-scale machines such as the power generation turbine consume a large amount of lubricating oil. Replacement of the lubricating oil interrupts operations of the machine to cause disadvantages such as generation loss and interruption of manufacturing. Furthermore, associated costs are required for purchase and delivery of new oil, oil replacement work, and waste disposal. It is desirable to prolong the useful lifetime of the lubricating oil as long as possible. Refrigerants for electric vehicles and data center undergo the oil diagnosis, based on which the refrigerant is replaced or hardware is repaired. The insulating oil for the transformer is also managed by performing the oil diagnosis.

From the recent carbon neutral perspective, electrification of automobiles that consume a large amount of petroleum-derived fuel is expected to decrease the demand for fuel from now onward. In many cases, however, there are no alternatives to bearings and gears that consume industrial oil. They are required to minimize oil consumption by prolonging the oil replacement period. By reducing oil consumption, carbon dioxide emissions may be decreased. Overlooking of deterioration and contamination of oil, however, may lead to machine failure.

In performing diagnosis of the lubricating oil property, “deterioration” and “contamination” are defined and distinguished from each other. The diagnosis is performed with respect to two broad categories including (1) oxidative deterioration of the lubricating oil with time, and (2) contamination of the lubricating oil with an external contaminant such as water, dust, and abrasion powder.

(1) Deterioration induced by oxidation of base oil, and deterioration induced by consumption of additives may be the oxidative deterioration of the lubricating oil. The oxidative deterioration of the lubricating oil may cause lowering of wear resistance, change in viscosity and the viscosity index, lowering of rust preventiveness, and lowering of anti-corrosiveness. As a result, the deterioration may accelerate wear of amplifier and fatigue of materials.

Although the lubricating oil is required to be useful for the period as long as possible, it is necessary to perform oil replacement and inspection of the device immediately in response to abnormal deterioration or abnormal contamination.

In the oil diagnostic technique, concerning change in transmittance of three wavelengths of RGB (red, green, blue) derived from the RGB sensor among visible light rays, as Patent Literature 1 discloses, there is a method for diagnosing the lubricating oil replacement time, and the sign of the machine from the relative change from the transmittance of new oil. The document discloses that the diagnosis is performed based on change in a value B among those of RGB, which has the largest change in the transmittance owing to oil deterioration.

In Patent Literature 2, a correlation of concentration between amine and quinone is focused, and discloses the technique for measuring the concentration of amine from chromaticity utilizing the correlation.

Conventionally, concerning monitoring of the lubricating oil condition based on color, the oil collected from the machine is sent to an analyzing company for instrumental analysis. Currently, the oil analysis has been still performed in this manner. In many cases, simple analytical operations have been performed on site, laboratories, or factories without entrusting the oil analyzing company with the analysis. Especially, as the color is an apprehensible index, engine oil, gear oil, turbine oil, operating oil, general-purpose machinery oil, bearing oil, compressor oil, or refrigerator oil has been visually diagnosed, or diagnosed visually by utilizing the standard color scale such as an ASTM color expression.

As it is well known, the ASTM color is expressed as a numerical value indicating the hue of the petroleum product such as the lubricating oil. The depth of the hue is expressed by comparison between the sample in the prescribed test tube and the standard color glass. The color is classified into 16 stages from the lighter color at an interval of 0.5. Color of the lubricating oil generally changes sequentially from colorless, yellow, reddish brown, and brown. According to the conventional technique, oil is replaced in response to color transition in two or more stages on the color scale.

Besides the ASTM color for the technique, a Saybolt color is used as the standard applied to the petroleum product. The test method applied to the Saybolt color is specified by JIS K 2580 as well as the ASTM color. The Saybolt color is expressed in 47 stages from +30 (bright) to −16 (dark).

### CITATION LIST

### Patent Literature

Patent Literature 1: Japanese Unexamined Patent Application Publication No. 2019-078718
Patent Literature 2: Japanese Unexamined Patent Application Publication No. 2021-015365

### SUMMARY OF INVENTION

### Technical Problem

Conventional art has problems to be described below. In the case where the lubricating oil color is visually determined using the ASTM color scale or the Saybolt color scale, determination results may differ from one another depending on the determiner.

In the method using apparatuses for ASTM color measurement, measurement results of the respective apparatuses largely differ. The large and expensive apparatus causes the problem of difficulties in continuous monitoring. The large-scale machine in operation generates vibration. In general, as the analyzer is susceptible to vibration, such machine is not suitable for continuous monitoring. As collected sample is required to be measured, it is impossible to perform direct measurement from a distant place.

An optical sensor having a white LED light source and an RGB sensor is an example of the sensor suitable for remote and continuous monitoring of the lubricating oil. The use of the optical sensor allows acquisition of color-related data, and continuous oil monitoring from the remote site via a network. Output values of the RGB sensor, however, are not color coordinates as specified by the standard.

Because of specification of the RGB sensor model, and manufacturing irregularity among semiconductor lots for sensor, there is no guarantee to consistently acquire the signal based on the same standard. That is, upon evaluation of oil based on data derived from the RGB sensor, the common scale cannot be shared among a plurality of users.

A method for evaluating oil deterioration is implemented by standardizing RGB values of new oil to be expressed as (255, 255, 255) in 8 data bits, or (100, 100, 100) by percentage indication, and performing evaluation in accordance with a relative change from the new oil. There are quite a few cases where the new oil is already colored. Accordingly, it is inconvenient to express such oil by the color system that specifies an absolute value of color degree, for example, by the ASTM color value.

In recent years, oil sensors of several types have been spreading. Specifically, examples of such sensor include a viscosity sensor using color and MEMS (Micro Electro Mechanical Systems), dielectric constant, conductivity, and infrared light. Upon oil sensor diagnosis using learning data like accumulated past data, for example, visually observed color change, ASTM color scale, viscosity analysis conducted by the oil analyzing company, and total oxidation analysis, parameters that can be measured by the oil sensor, for example, the dielectric constant and conductivity, have to be indirectly estimated with respect to viscosity and total oxidation by a data analysis technique such as machine learning. This causes a reliability problem in isolation of cause of the change. The color scale measurement data based on color derived from visual observation and the apparatus can be easily utilized for evaluation of oil properties.

It is an object of the present invention to provide consistent standard values for evaluating oil properties using the optical sensor.

### Solution to Problem

An oil diagnostic method according to an aspect of the present invention includes: storing, in a storage device, a calibration curve based on first data and second data, the first indicating, as a color scale value, a color change of a standard substance having a known color scale value, and the second data indicating a color change based on a result of measuring the standard substance by using an optical sensor; acquiring, from an input device, third data based on a result of measuring oil having unknown color coordinates by using the optical sensor; and calculating, by using a processing device, a color scale value corresponding to the color coordinates of the oil from the third data by referring to the calibration curve.

According to another aspect of the present invention, the oil diagnostic system is configured to use an optical sensor having a light source and a light receiving element to transmit visible light rays from the light source through oil, and executes processing of chromaticity information corresponding to three different wavelengths obtained by detecting the visible light ray transmitted through the oil by using the light receiving element. The oil diagnostic system includes: an input device that acquires the chromaticity information; a storage device that stores a calibration curve based on first data and second data, the first data indicating, as a color scale value, a color change of a standard substance having a known color scale value, and second data indicating a color change based on a result of measuring the standard substance by using the optical sensor; and a processing device that converts the chromaticity information into the color scale value by referring to the calibration curve.

### Advantageous Effects of Invention

The use of the optical sensor allows provision of consistent standard values for evaluating oil properties. Objects, configurations, and effects other than the above will be apparent from the description of the following embodiments.

### BRIEF DESCRIPTION OF DRAWINGS

FIG. 1 is a graph representing change in color caused by the use of gear oil.

FIG. 2 is a graph representing spectral-sensitivity characteristics of an RGB color sensor.

FIG. 3 is a graph representing change in color of oil caused by intrusion of water.

FIG. 4 is a view representing a calibration curve for conversion from a ΔE value to a color scale value.

FIG. 5 is a view representing a calibration curve for conversion from a B value to a color scale value.

FIG. 6 is a view representing a calibration curve for conversion from a ΔE value to a color scale value.

FIG. 7 is a view representing a calibration curve for conversion from a B value to a color scale value.

FIG. 8 is a graph representing diagnostic results of amplifier oil, which have been derived from an optical sensor.

FIG. 9 is a graph representing a method for extracting an abnormal sample owing to contamination from diagnostic results of oil, which have been derived from the optical sensor.

FIG. 10 is a flowchart representing process steps of measuring the lubricating oil by the optical sensor, and converting the measurement results into the color scale, and outputting the color scale.

FIG. 11 is a block diagram representing an oil monitoring system according to an embodiment.

FIG. 12 is a block diagram representing an oil monitoring system according to another embodiment.

### DESCRIPTION OF EMBODIMENTS

Referring to the drawings, an embodiment is described in detail. It is to be noted that the present invention shall not be construed as being limited by description of the embodiment as below. A person skilled in the art can easily understand that the specific configuration is variable so long as the variation does not deviate from the idea and meaning of the present invention.

In the configuration of embodiments as described below, components having identical parts or similar functions are denoted by the same signs throughout different drawings, and overlapped explanations may be omitted.

A plurality of elements each having the same or similar function may be described by attaching different subscripts to the same signs. Explanations may be made while omitting the subscript in the case where those elements do not have to be distinguished from one another.

Such description in the specification as “first”, “second”, “third” is intended to identify the component, and does not necessarily limit the number, order, or the content. The number for identifying the component is used for each context. The number used in one context does not necessarily indicate the same configuration in another context. The component identified by the specific number may be imparted with the function of the component identified by another number.

For easy understanding of the present invention, the position, size, shape, and range of each configuration as illustrated in the drawings do not necessarily express actual position, size, shape, and range. Accordingly, the present invention is not necessarily limited by the position, size, shape, and range as disclosed in the drawings.

Publications, patents, and patent applications as cited in this specification form a part of descriptions of the specification.

In the specification, the component expressed in the singular may also contain the one expressed in the plural unless otherwise clarified in the specific context.

The machine monitoring method utilizing color change in the lubricating oil as described in the following embodiments in detail is implemented by converting measurement values derived from the optical sensor having the RGB sensor into standard values such as the ASTM color scale and the Saybolt color.

The use of the RGB sensor allows continuous remote oil monitoring. Conversion of outputs from the RGB sensor into standard values like the ASTM color scale results in output of standard values that allow consistent evaluation of oil properties irrespective of the sensor type. Even in the case of different oil types and different types of oil-consuming machines, standard values which allow consistent evaluation of oil properties are output. It is therefore possible to assure compatibility of data among measurers, and to utilize past data. The ground on which the above-described advantages are attained is described below.

The lubricating oil is composed of base oil and an additive agent. The base oil is a comparatively chemically stable organic compound such as a hydrocarbon and ether, and the additive agents are mostly organic compounds. Accordingly, most of the components of the lubricating oil are organic compounds. Generally, oxidative deterioration induced by the use of the lubricating oil may be considered as the oxidative deterioration of the organic compound. It is known that the oxidative deterioration of the organic compound intensifies the tone of color in the range from yellow to reddish brown. Therefore, deterioration in the lubricating oil is diagnosed by the ASTM color scale. Consequently, the color change is induced by the use of the lubricating oil in the similar manner irrespective of different type of lubricating oil. It is therefore possible to use the ASTM color scale for various types of oil

Machines that consume the lubricating oil include parts such as bearings and gears of different types, in different sizes, and having different rotating speeds. There are many machines which are different in size and function, for example, the amplifier for the wind turbine, reducers for railway vehicles, construction machinery, automobiles, and generators. Apart from different operation temperatures, the lubricating oil generally undergoes oxidation regardless of the type and size of the machine as it is used for reducing the friction on the sliding surface, or preventing the wear. Accordingly, the ASTM color scale can be similarly used irrespective of the machine type.

### <Change in Oil Property>

Types of lubricating oil include engine oil, turbine oil, hydraulic oil, bearing oil, sliding surface oil, gear oil, compressor oil, cutting oil, and the like. The lubricating oil is composed of base oil and an additive agent. Examples of the additive agent include an antioxidant, rust-preventive agent, antifoaming agent, viscosity index improver, oiliness improver, extreme-pressure additive, detergent dispersant, pour point depressant, and an emulsifier. The transformer oil is used as oil for usage except lubrication. For most types of oil, change in color of oil in use becomes an index for determination with respect to the replacement time and machine abnormality.

The lubricating oil is composed of base oil and an additive agent. Examples of the base oil include petroleum-derived mineral oil, high-performance synthetic oil, and plant-derived bio-oil. The synthetic oil is of high purity, chemically highly stable, and unlikely to be deteriorated. In most cases, color of the lubricating oil produced by using the synthetic oil changes owing to consumption of the additive agent. Meanwhile, the use of the mineral oil or the bio-oil having low purity may colorize the base oil because of an ester structure that is slightly chemically unstable. Colorization is also caused by consumption of the additive agent.

Color of oil will change, specifically, the new oil that is colorless or in light yellow has its color changed as passage of several days for use from yellow, orange, reddish brown, and blackish brown. The color change allows the color scale measurement data derived from visual observation and the apparatus to be utilized for the oil sensor.

### First Embodiment

### <Color Change Induced by Deterioration>

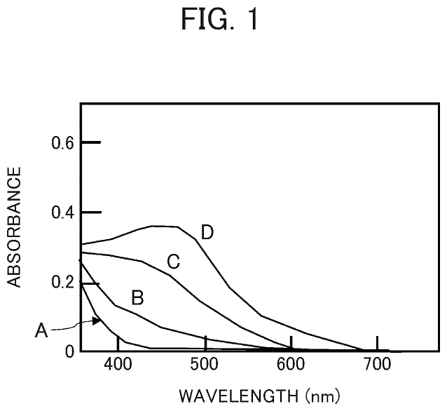

FIG. 1 is a view representing color change caused by the use of gear oil. Each curve denotes visible absorbance of (A) new oil, (B) the oil used for two months, (C) the oil used for six months, and (D) the oil used for one year, respectively. The appearance color of the new oil is slightly yellowish but substantially colorless and transparent. The color of the oil (B) is light yellow. Each color of the oil (C) and (D) is orange and reddish brown, respectively. Each color of the engine oil, turbine oil, hydraulic oil, bearing oil, sliding surface oil, compressor oil, cutting oil, rolling oil, and insulating oil also changes similarly.

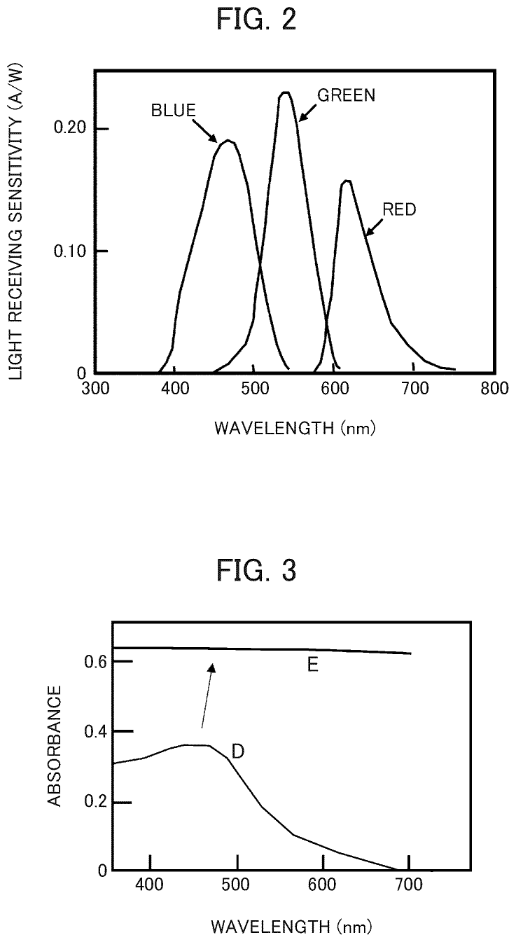

FIG. 2 represents spectral sensitivity characteristics of the RGB color sensor formed of a Si photo diode array. A 3ch (RGB) photo diode having each sensitivity for Blue (λp=460 nm), Green (λp=540 nm), Red (λp=620 nm) is used. This color sensor exhibits the spectral sensitivity characteristic resembling visibility. The use of this color sensor allows the oil color to be expressed as color coordinates.

### <Color Change Induced by Contamination>

FIG. 3 shows an example (E) indicating that the gear oil (D) used for a year is contaminated with intruded water. The gear oil (D) is transparent and colored in reddish brown. The gear oil (E) is cloudy and brown colored owing to contamination with water at 1 wt %. In this case, a rise in the absorbance, that is, decrease in transmittance occurs over the entire wavelength region in the visible range.

The absorbance and the transmittance are defined as below. In the case where light is transmitted through the sample having a uniform optical path length,

$$absorbance = log ( Ii / Io ) , and$$

$$transmittance ( \% ) = ( Io / Ii ) * 100 ,$$

where Ii denotes intensity of incident light, and Io denotes intensity of transmitted light.

As described above, the oil property can be determined based on change in color. The use of data derived from the RGB color sensor allows remote oil monitoring by omitting sample collection on site.

Because of specification of the RGB color sensor model, and manufacturing irregularity among semiconductor lots for sensor, there is no guarantee to consistently acquire the signal based on the same standard. Basically, output values of the RGB color sensor are not based on the standard, but are mere voltage signals of RGB, which rely on the sensor sensitivity.

In embodiments to be described below, an optical sensor is used to preliminarily prepare a calibration curve in accordance with the standardized color scale such as the ASTM standard color. The lubricating oil is then measured under the above-described condition. This makes it possible to convert the RGB outputs of the optical sensor into the color scale. The ASTM color scale has been generally employed as the standardized color scale. This color scale is available in many situations because of compatibility with data acquired in the past using the ASTM color scale.

### <Setting of Reference Value Using Transparent Sample>

As the RGB type optical sensor is used for single beam type measurement, the reference value has to be set before starting the measurement. The standard glass or standard solution with known color coordinates is employed for preparing the calibration curve for color scale conversion. It is therefore necessary not to use new oil but use such solvent as pure water having 100% visible light transmittance, methanol and acetone for setting the reference value.

In an exemplary case, the optical sensor is used for measurement of bubble-free pure water to set measurement values to (255, 255, 255). In the lubricating oil measurement, a relative value to the measurement value of pure water is obtained. Alternative to the pure water, it is possible to use an organic solvent with 100% visible light transmittance such as ethanol, acetone, toluene, and xylene.

### <Calibration Curve Prepared Using ASTM Standard Glass or Standard Solution Sample>

As specified by ASTM D1500, the ASTM color is obtained by classifying color of the petroleum product, which has been converted into numerical value in the range from 0.5 as light color to 8.0 as dark color. The ASTM color standard glass is compared with the sample so that the numerical value of the color is expressed by the number of the color standard glass, which is substantially equivalent to the sample.

The numerical values are classified at an interval of 0.5. If the sample color is in between two ASTM colors, a letter L is prefixed to the numerical value indicating the darker color. For example, if the sample color is in between 4.5 and 5.0, the value is expressed by L5.0. The sample color that is darker than 8.0 is expressed by D8.0.

TABLE A1.2 in the ASTM D1500-07 defines color coordinates of the ASTM color values, and Luminous Transmittance values. The ASTM standard glass is the glass in color that satisfies the color coordinates and specification of Luminous Transmittance. Besides the ASTM standard glass, color standard solution samples of four types including A1, A3, A5, A7 are available.

The following describes the process for preparing the calibration curve in order to convert the optical sensor output into the ASTM color scale. The ASTM standard glass is placed at an oil measurement position of the optical sensor to perform measurement of transmitted light. As the ASTM color is classified into 16 stages, the 16-staged standard glasses are measured sequentially.

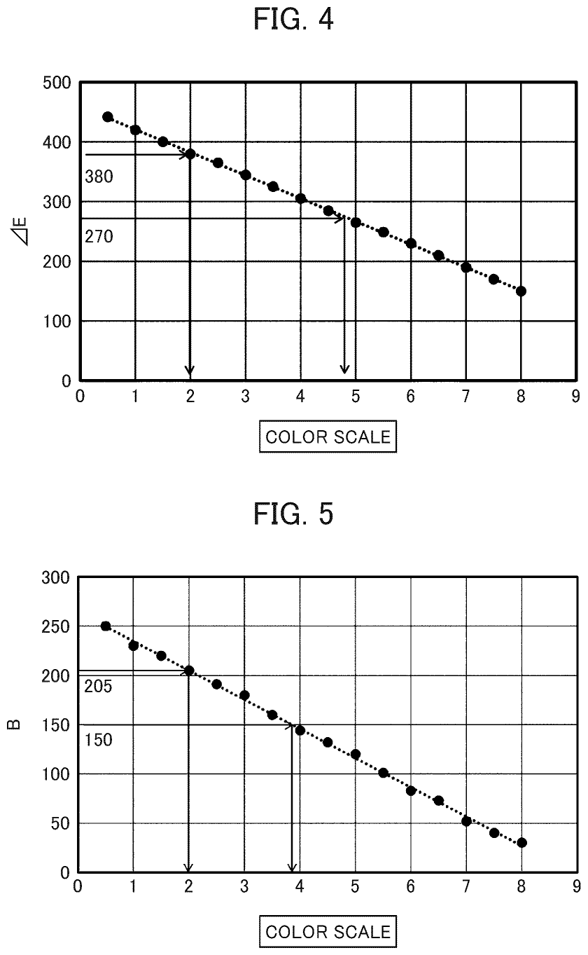

FIG. 4 is a graph representing a relationship between a ΔE and the ASTM color scale, which is usable as the calibration curve. The calibration curve is prepared as below. The equation ΔE=√(R²+G²+B²) can be solved from RGB output signals of the RGB color sensor.

An initial value of the optical sensor is set (white setting) using the base oil or the like, having its transmittance in the visible range confirmed as 100%. FIG. 4 represents the ΔE values obtained by measuring the ASTM color standard glasses from 0.5 to 8.0 using the RGB color sensor.

The calibration curve indicating the correspondence between the ΔE value and the ASTM color scale as shown in FIG. 4 is preliminarily stored in the storage device and the memory. The RGB measurement values are obtained by measuring the oil color by the RGB color sensor to allow evaluation by the use of ΔE values. Referring to ΔE values and the calibration curve in FIG. 4, if the ΔE of deteriorated oil is 380, the color scale becomes 2. If the ΔE of another deteriorated oil is 270, the color scale is in between 4.5 and 5. In this case, as the larger value is selected, the color scale becomes 5. FIG. 4 is usable as the calibration curve.

FIG. 5 is a graph representing a relationship between a B value and the ASTM color scale, which is usable as the calibration curve. It can be prepared similarly to the calibration curve as shown in FIG. 4. In this case, the value B is used instead of the ΔE. Among RGB values, the value B exhibits higher correlations with both deterioration of oil and the color scale. If the value B of deteriorated oil is 205, the color scale becomes 2. If the value B of the deteriorated oil is 150, the color scale is in between 3.5 and 4. The color scale then becomes 4.

The liquid with 100% transmittance in the visible range is available for white setting. Examples of such liquid include water, organic solvent, fuel, and base oil.

### <ASTM Standard Solution>

Besides the ASTM color standard glass, the ASTM standard solution for calibrating the ASTM color measurement device allows preparation of the calibration curve. The ASTM standard solution has four types including 1, 3, 5, 7. Other types of standard solution corresponding to 2, 4, 6, 8 can be prepared by changing the reagent concentration and dilution.

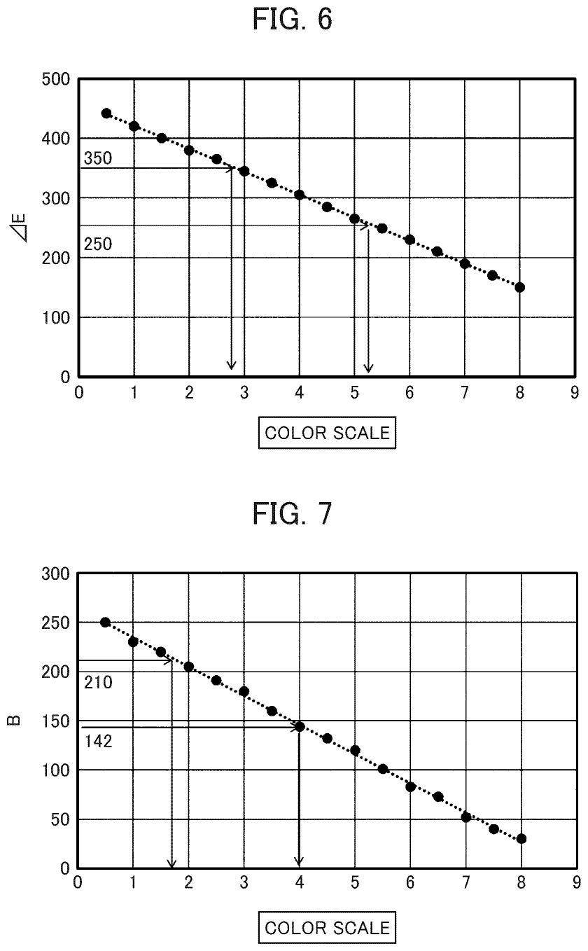

FIG. 6 is a graph representing a relationship between a ΔE and the ASTM color scale, which is usable as the calibration curve. The calibration curve is prepared as below.

The initial value of the optical sensor is set (white setting) using the organic solvent having its transmittance in the visible range confirmed as 100%. FIG. 6 represents values derived from measurement of the ASTM standard solution adjusted in accordance with the ASTM color ranging from 0.5 to 8 at an interval of 0.5 by using the RGB color sensor. The RGB measurement values may be evaluated using the ΔE values similarly to the case as shown in FIG. 4.

Referring to the calibration curve as shown in FIG. 6, if the ΔE of deteriorated oil is 350, the color scale value is in between 2.5 and 3. The color scale then becomes 3. If the ΔE of another deteriorated oil is 250, the color scale value is in between 5 and 5.5. In this case, as the larger value is selected, the color scale becomes 5.5. FIG. 6 is usable as the calibration curve.

FIG. 7 is a view representing a relationship between the color scale and the value B. The ASTM standard solution is measured by the RGB color sensor similarly to the case as shown in FIG. 6. If the value B of deteriorated oil is 210, the color scale becomes 2. If the value B of deteriorated oil is 142, the color scale becomes 4. Similar to the case as shown in FIG. 5, FIG. 7 is usable as the calibration curve.

### <Detection of Contamination>

Change in the lubricating oil as it is used is induced by deterioration and contamination. The deterioration represents change in quality of the lubricating oil itself. Meanwhile, there may be cases where any one of water, coolant, microparticle, and abrasion powder intrudes into the lubricating oil used for machines. It is possible to distinguish between deterioration and contamination using values of ΔE and MCD, which have been derived from measurement values of the RGB optical sensor. The MCD value is the difference between the maximum and minimum values of three RGB values.

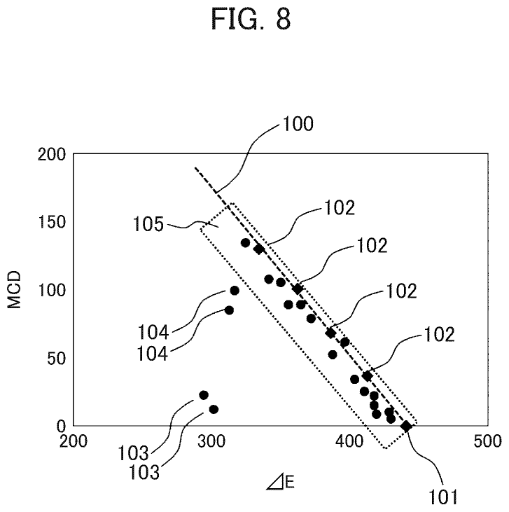

FIG. 8 represents a map indicating change in oil color induced by the use of amplifier oil (gear oil) for the wind turbine. Referring to the graph in FIG. 8 representing a relationship between values of ΔE and MCD, new oil denoted by 101 changes its color as it is used for the amplifier in the left upward direction.

A reference numeral 102 denotes a plot of a forcibly deteriorated oil sample. A deterioration curve 100 is an approximate curve derived from values of the forcibly deteriorated sample. As this curve indicates the color change induced by deterioration of oil, it is called a deterioration curve. Normal oil samples are distributed in a frame denoted by a reference numeral 105. Each sample 104 having intruded microparticles and metal abrasion powder is plotted outside the frame because of decrease in all the RGB values. The water intruded sample becomes cloudy white to largely decrease the visible light transmittance. It is plotted at a position 103 greatly apart from the frame 105.

It is preferable to extract and remove abnormal samples 103 and 104 as described above before conversion of the RGB values of the optical sensor into the ASTM color scale values.

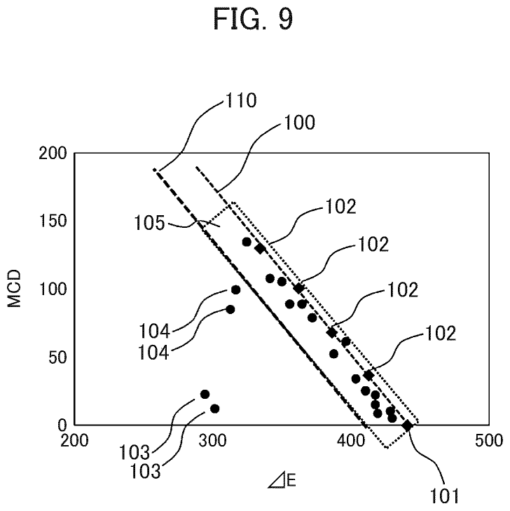

A method for extracting abnormal samples is described referring to FIG. 9. If the deterioration curve 100 is approximated with a linear expression, such curve can be expressed by the equation of MCD=−a×ΔE+b.

The sample plotted on the left side of the curve 110 is determined as the abnormal sample. The curve 110 is expressed as MCD=−a×ΔE+c.

A relationship between terms b and c may be expressed as b>c.

That is, in the case of MCD<−a×ΔE+c, the subject sample is determined as the abnormal sample.

### <Diagnostic Flow and System Configuration>

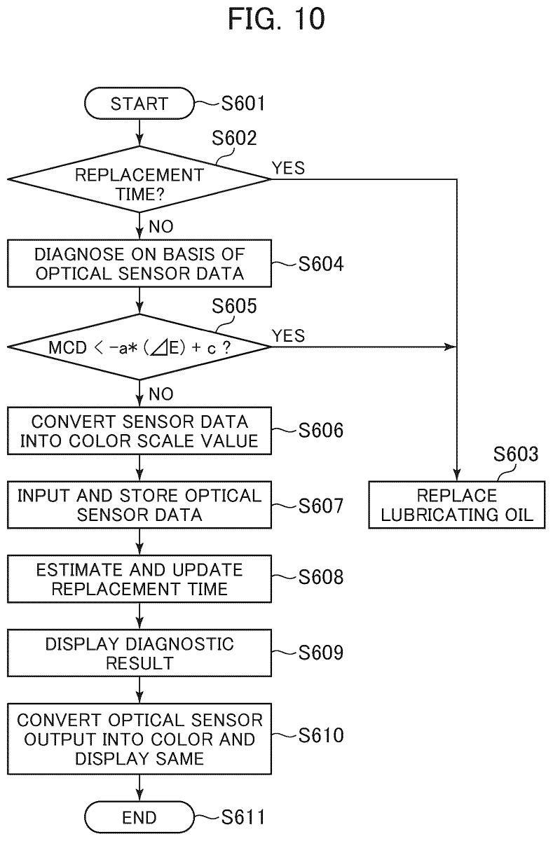

FIG. 10 represents a processing flow including process steps of diagnosing both deterioration and contamination of the lubricating oil, converting the normal sample value into the ASTM color scale value, and outputting the result.

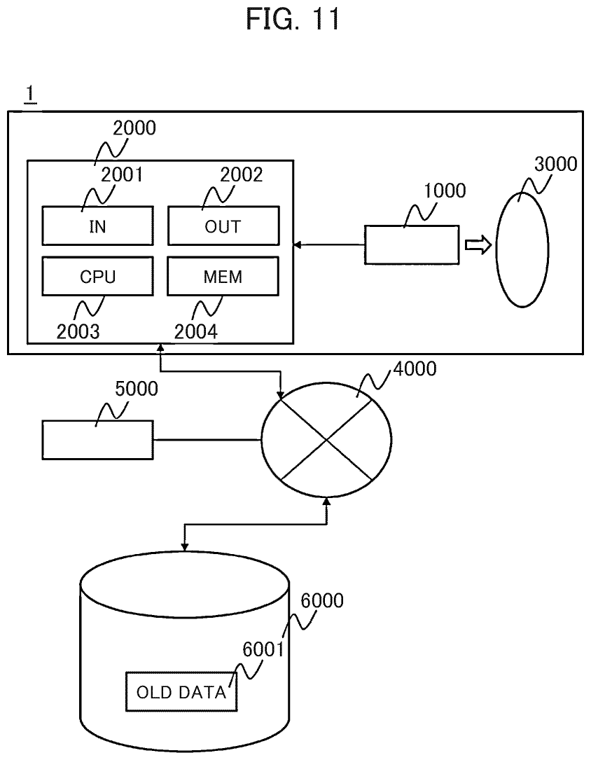

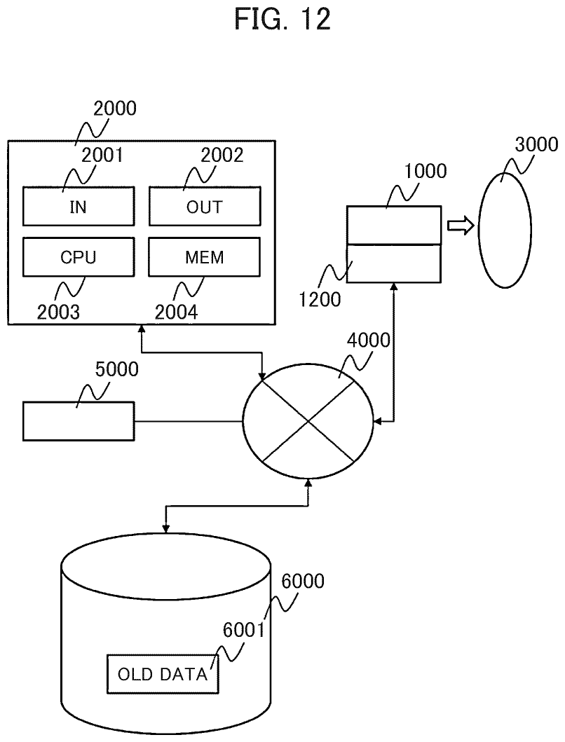

FIG. 11 is a block diagram of a system that executes the processing flow as represented by FIG. 10. Basically, an oil monitoring system 1 is configured by providing an RGB optical sensor 1000 (commercial product is available) with a microcomputer 2000. It is possible to prepare a product as an optical sensor formed by integrating the RGB optical sensor 1000 and the microcomputer 2000. The microcomputer 2000 includes an input device 2001 (IN), an output device 2002 (OUT), a processing device 2003 (CPU), and a storage device 2004 (MEM). Description of an internal bus for connecting those devices is omitted.

An example of remote monitoring of oil 3000 by the present system is described. The microcomputer 2000 is communicable with a remote server 5000 via the input device 2001, the output device 2002 and a network 4000.

The processing is executed as represented by FIG. 10. The calibration curve as described referring to FIGS. 4 to 7 is preliminarily stored in the storage device 2004. It is preferable to prepare the calibration curve by the RGB optical sensor 1000 itself, or the sensor with the same specification or of the same model as the sensor 1000.

S601: Execution of the processing is started at the predetermined time stored in the storage device 2004, or any time instructed from the server 5000.

S602: The processing device 2003 determines whether the time has reached the predetermined replacement time stored in the storage device 2004.

S603: If the time has reached the replacement time, the processing device 2003 notifies the server 5000 of the need of replacement of the lubricating oil.

S604: If the time has not reached the replacement time, the processing device 2003 instructs the RGB optical sensor 1000 to perform optical measurement of oil. The RGB optical sensor 1000 acquires RGB signals, and sends data to the microcomputer 2000. The data are received by the input device 2001, and stored in the storage device 2004. At this time, the data may be transmitted to the server 5000 from the output device 2002.

S605: The processing device 2003 determines with respect to a sign of contamination based on the principle as shown in FIG. 9. It is assumed that the data representing the curve 110 and the deterioration curve 100 as shown in FIG. 9 are preliminarily stored in the storage device 2004. The processing device 2003 calculates values of ΔE and MCD from the RGB signal, and makes a determination based on the formula MCD<−a×ΔE+c. Processing is executed by the processing device 2003 as below. The processing device 2003 may be configured to execute the program stored in the storage device 2004, or configured to be hard-wired to implement similar functions.

If there is a sign of contamination, the processing device 2003 notifies the server 5000 of the need of replacement of the lubricating oil (S603).

S606: If there is no sign of contamination, the processing device 2003 converts the RGB signal into the color scale value. For this, the storage device 2004 preliminarily stores data of calibration curves as shown in FIGS. 4, 5, 6, 7. The processing device 2003 performs conversion into the ASTM color scale value with reference to data of calibration curves. The conversion may be performed based on the ΔE, or the value B.

S607: The converted ASTM color scale value is stored in the storage device 2004. The color scale value may be transmitted to the server 5000. The data transmission to the server may be performed in real time or by batch processing.

S608: The processing device 2003 performs diagnosis such as estimation of the oil replacement time based on the color scale value or the RGB signal. In this case, for example, the RGB signals acquired by the RGB optical sensor 1000, or values B or ΔE derived from such signals are stored in the storage device 2004 in time series so that those values are monitored as the time series data. In this manner, the oil condition is diagnosed and estimated. The estimation method is exemplified as disclosed in Patent Literatures 1 and 2.

S609: The processing device 2003 transmits data of diagnostic results to the server 5000 for displaying the diagnostic results.

S610: The server 5000 may be configured to display data of diagnostic results as color data based on the RGB signals, or color scale values.

S611: When the server 5000 confirms data reception, the processing ends.

The use of the lubricating oil diagnostic flow as shown in FIG. 10 allows diagnosis of both deterioration and contamination of the lubricating oil. If the sample is normal, it is possible to perform conversion into the ASTM color scale value, and output the results. This diagnostic method is applicable to general types of oil except the amplifier oil for wind turbine.

The Saybolt color scale is applicable to preparation of calibration curves by similar procedures. Furthermore, the color scale standard for products other than petroleum products such as the lubricating oil is also applicable by similar procedures.

This embodiment describes an exemplary case where the optical sensor is integrated with the oil monitoring system 1 installed on the site where the oil consuming machine is placed. The optical sensor (oil monitoring system 1) has functions installed therein for converting an output of current or voltage from the RGB optical sensor 1000 into a digital signal, further converting the signal into the color scale value, and accumulating such value. The storage device 2004 stores the calibration curve data for conversion of the lubricating oil measurement value into the color scale value. After conversion into the color scale value in the optical sensor, the converted data are transmitted to the computer for diagnosis such as the server 5000 by the digital cable or wireless LAN. Alternatively, the converted color scale value may be sent to the server on the cloud from the optical sensor.

Data of calibration curves in the storage device 2004 may be appropriately changed or updated via the network 4000 from the server 5000.

A database 6000 connected via the network 4000 stores old data 6001 based on the ASTM color scales accumulated in the past. The color information measured by the RGB optical sensor of the oil monitoring system 1 is compatible with the legacy ASTM color scale. It is therefore possible to hold continuity with past data.

### Second Embodiment

### <Example of Remote Monitoring>

An optical sensor capable of outputting RGB values of oil is mounted on an oil collection port disposed at the lower part of an amplifier of 2 MW wind turbine. The optical sensor data are collected by a data logger, and collected data are transmitted to a monitoring server via a wind turbine LAN.

The optical sensor continuously measures color of the amplifier oil for the wind turbine. ASTM color scale values are derived from the RGB measurement values to output results to a monitor of the wind turbine.

Concerning maintenance of industrial oil such as lubricating oil, insulating oil, machining oil by the color measurement process for measuring three primary colors, color measurement results are converted into the color scale to be output. The use of the RGB optical sensor allows accurate grasping of the ASTM.

### Third Embodiment

In the first embodiment, the oil monitoring system 1 is installed on the site where the oil consuming machine is placed so that conversion into the color scale is performed in the oil monitoring system 1.

In another example, output signals of current or voltage from the RGB optical sensor 1000 are converted into digital signals by the computer through analog cables to allow output of color scale values using the calibration curve data stored on the computer or a cloud.

FIG. 12 represents a structure of another oil monitoring system. An interface 1200 digitizes a signal of the RGB optical sensor 1000 by a known method so that the digitized signal is transmitted to the microcomputer 2000 (larger scale computer such as a server is available) through the network 4000 connected thereto. The microcomputer 2000 generates the color scale value by executing the similar processing to the one in the first embodiment.

### Fourth Embodiment

In the above-described embodiments, conversion into the ASTM color scale value is described as an example. It is also possible to perform conversion into other color scales. Besides the color scale for petroleum products as specified by the ASTM D-1500, values measured by the optical sensor having the RGB sensor can be converted into the color scale such as APHA, HAZEN, Pt-Co, Saybolt color, and Gardner.

The HAZEN unit color number is an index for evaluating a color degree of a chemical product in a liquid state at room temperature, or a chemical product to be melted by heating. It is called “platinum-cobalt color scale” or “APHA color”. A value of color of 1 liter of solution that contains 1 mg of platinum of hexachloroplatinic ion form and 2 mg of cobalt(II)chloride hexahydrate is set to 1 as the index. It is described in Section 1 of JIS K0071-1 Color test method of chemical products. The method is implemented for quantifying the degree of yellowness of the highly transparent substance.

The optical sensor having the white LED and the RGB sensor is used to measure color coordinates of commercial diesel fuel. The measured value is imported to the PC as the voltage output, and converted into a digital value. Using calibration curve data on the basis of the HAZEN unit color number, which have been preliminarily stored in the PC, the diesel fuel color is converted into the HAZEN color. The resultant HAZEN color value is 2.

### Fifth Embodiment

Like the ASTM color, the Saybolt color is employed for expressing color of the petroleum product. The ASTM color expresses colors from light color to dark color in a range between 0.5 and 8.0 (at an interval of 0.5). Meanwhile, the Saybolt color is obtained by classifying color of the transparent liquid into stages from +30 as the brightest color value to −16 as the darkest color value (at an interval of 1).

The optical sensor having the white LED and the RGB sensor is used for color measurement of refined fuel. Color of distillated fuel is measured by the optical sensor having the white LED and the RGB sensor, in which a memory for conversion into the color scale and a microcomputer for arithmetic operation are installed to output Saybolt color scale values.

The use of this sensor and the Saybolt color scale output from the sensor allows evaluation of the sulfur removing rate of diesel fuel, quality of liquefied natural gas, and color of the lubricating oil product.

### Sixth Embodiment

A Gardner color is a scale to be used for measuring the degree of yellowness. The Gardner color scale for measuring the high density yellow and the HAZEN color scale for measuring the low level yellow overlap each other.

The optical sensor having the white LED and the RGB sensor is used for color measurement of engine oil. Color of engine oil is measured by the optical sensor having the white LED and the RGB sensor, in which a memory for conversion into the color scale and a microcomputer for arithmetic operation are installed to output Gardner color scale values.

### Seventh Embodiment

An EBC color is a color scale for evaluating color of beer. The beer having the EBC value ranging from 0 to 15 is called Pale Beer. The beer having the EBC value ranging from 0 to 35 is called Dark Beer. The optical sensor having the white LED and the RGB sensor is used for color measurement of Japanese beer. Color of beer is measured by the optical sensor having the white LED and the RGB sensor, in which a memory for conversion into the color scale and a microcomputer for arithmetic operation are installed to output EBC color scale values.

An ASBC as the color scale for beer is applicable to the measurement to be similarly performed.

An ICUMSA color scale is used for saccharide solution. The optical sensor having the white LED and the RGB sensor is used for color measurement of muscovado. Color of muscovado is measured by the optical sensor having the white LED and the RGB sensor, in which a memory for conversion into the color scale and a microcomputer for arithmetic operation are installed to output ICUMSA color scale values.

A Lovibond color has been used for various markets of oil and fat processed food, chemical products manufactured in chemical plants, petroleum products, and other products. The optical sensor having the white LED and the RGB sensor is used for color measurement of olive oil. Color of olive oil is measured by the optical sensor having the white LED and the RGB sensor, in which a memory for conversion into the color scale and a microcomputer for arithmetic operation are installed to output Lovibond color scale values.

As described in the foregoing embodiments, in the diagnostic operation of liquid having unknown color coordinates such as the petroleum product, the color scale value of such liquid having unknown color coordinates may be obtained using a first result indicating change in color of the liquid having unknown color coordinates, which is obtained based on an output value of the optical sensor, a second result indicating change in color of liquid having a known color scale value, which is obtained based on the output value of the optical sensor, and a preliminarily acquired correlation function or a correspondence table between the first and the second results.

Advantageous effects of the embodiments allow accumulated past data of color scales to be utilized for oil diagnosis using the RGB optical sensor. Furthermore, an online color scale measurement can also be performed.

According to the embodiments, as the oil consumption is reduced, the carbon emission amount is decreased to contribute to prevention of global warming, and attainment of sustainable society.

1. An oil diagnostic method comprising the steps of:
storing a calibration curve based on first data and second data in a storage device, the first data indicating, as a color scale value, a color change of a standard substance having a known color scale value, and the second data indicating a color change based on a result of measuring the standard substance by using an optical sensor;
acquiring third data from an input device, the third data being based on a result of measuring oil having unknown color coordinates by using the optical sensor; and
calculating, by using a processing device, a color scale value corresponding to the color coordinates of the oil from the third data by referring to the calibration curve.

2. The oil diagnostic method according to claim 1, wherein,
an RGB sensor is used as the optical sensor, and
change in a wavelength B is used as the color change indicated by the second data.

3. The oil diagnostic method according to claim 1, wherein,
an RGB sensor is used as the optical sensor, and
change in a ΔE value is used as the color change indicated by the second data.

4. The oil diagnostic method according to claim 1, wherein
a condition of the oil is determined by monitoring the third data as time series data.

5. The oil diagnostic method according to claim 1, wherein
the oil is lubricating oil used for a machine.

6. The oil diagnostic method according to claim 1, wherein
the color scale value is an ASTM color scale.

7. The oil diagnostic method according to claim 1, wherein
the color scale value is at least a color scale selected from a group including HAZEN, a Saybolt color, and Gardner.

8. The oil diagnostic method according to claim 1, wherein,
the third data acquired by the optical sensor is transmitted,
an information processor including the input device and the storage device receives the third data, and
the information processor acquires the color scale value corresponding to color coordinates of the oil from the third data.

9. The oil diagnostic method according to claim 1, wherein
in measurement of the second data by the optical sensor, an initial value is preliminarily set using a substance having its transmittance in a visible range confirmed as 100%.

10. An oil diagnostic system which is configured to use an optical sensor having a light source and a light receiving element to transmit visible light rays from the light source through oil, and executes processing of chromaticity information corresponding to three different wavelengths obtained by detecting the visible light ray transmitted through the oil by using the light receiving element, the oil diagnostic system comprising:
an input device that acquires the chromaticity information;
a storage device that stores a calibration curve based on first data and second data, the first data indicating, as a color scale value, a color change of a standard substance having a known color scale value, and the second data indicating a color change based on a result of measuring the standard substance by using the optical sensor; and
a processing device that converts the chromaticity information into the color scale value by referring to the calibration curve.

11. The oil diagnostic system according to claim 10, wherein
the optical sensor, the input device, the storage device, and the processing device are integrated to form an oil diagnostic apparatus.

12. The oil diagnostic system according to claim 10, wherein,
the chromaticity information is transmitted from the optical sensor to an information processor outside the optical sensor,
the information processor includes the input device, the storage device, and the processing device, and
based on the chromaticity information received by the input device, the processing device refers to the calibration curve to convert the chromaticity information into the color scale value.

13. The oil diagnostic system according to claim 10, wherein,
the optical sensor is an RGB sensor for outputting RGB signals, and
the calibration curve indicates a corresponding relation between a value B derived from the RGB signal and the color scale value.

14. The oil diagnostic system according to claim 10, wherein,
the optical sensor is an RGB sensor for outputting RGB signals, and
the calibration curve indicates a corresponding relation between a ΔE value derived from the RGB signal and the color scale value.

15. The oil diagnostic system according to claim 13, wherein
the color scale value is at least a color scale selected from a group including an ASTM color scale, HAZEN, a Saybolt color, and Gardner.
